(19) United States
(12) Patent Application Publication
(10) Pub. No.: US 2019/0308116 A1
(43) Pub. Date: Oct. 10, 2019

(54) Solvent based cannabinoid extraction process with improved efficiency, safety, quality, which yields a homogenous and pasteurized product

(71) Applicant: Craig Alan Brodersen, Eugene, OR (US)

(72) Inventor: Craig Alan Brodersen, Eugene, OR (US)

(21) Appl. No.: 15/949,072

(22) Filed: Apr. 10, 2018

(51) Int. Cl. B01D 11/04; B01F 3/08

(52) U.S. Cl. CPC B01D 11/0492; B01D 11/0484; B01D 11/0488; C07D 311/80; B01F 3/0815; B01D 2257/702; B01F 3/0811

(57) ABSTRACT

A solvent based cannabinoid extraction process and apparatus that retains the flavonoids and terpenes by using low temperature, high surface area inline filtration, vacuum distillation, homogenization and pasteurization to produce a superior finished product.

### RELATED U.S. APPLICATION DATA

Continuation of provisional patent application No. 62/484,400, filed on Apr. 12, 2017

### FIELD OF CLASSIFICATION SEARCH

A61K 36/185

B01D 11/027, 11/207, 11/219

C11B 1/10, 1/102, 1/104

References Cited,

| | | |
|---|---|---|
| 2,682,551 | June 1954 | Miller |
| 4,331,695 | May 1982 | Zosel |
| 5,041,245 | August 1991 | Benado |
| 5,281,732 | June 1994 | Franke |
| 5,405,633 | April 1995 | Heidlas |
| 5,525,746 | June 1996 | Franke |
| 5,739,364 | April 1998 | Franke |
| 6,248,910 | June 2001 | Franke |
| 6,676,838 | June 2004 | Corr |
| 8,859,793 | October 2014 | Hamler |
| 9,327,210 | May 2016 | Jones |
| 9,757,664 | September 2017 | McGhee |
| 9,789,147 | October 2017 | Jones |
| 9,844,740 | December 2017 | Jones |
| US 2017/033350 | November 2017 | Ayres |

### OTHER PUBLICATIONS

http://www.tamisiumextractors.com/

https://bhogart.com/product-category/extractors/extraction-kits/

https://precisionextraction.com/wp-content/uploads/2018/03/2018.03.08.X10-Manual-FD.pdf

### BACKGROUND

Extraction of cannabinoids using solvents is a fairly recent practice. The use of solvents that are vapors at ambient or room temperatures is one of the more widely used approaches. The systems on the market are fairly simple designs that can be very time consuming if operated for optimal safety. Also the solvents tend to be subjected to thermal changes for different reasons during the process that are also time consuming. What they do not teach is product homogenization, pasteurization, or how to remove the residual solvents from the extract and quality control sample acquisition.

The use of solvents to extract oils from plant and animal products has been done for many years. The use of liquefied petroleum gas (LPG), has been suggested as long ago as 1928 by Reid, U.S. Pat. No. 1,802,533. In more recent years work has been done on solvent based cannabinoid extraction. Generally pressure is used to keep solvents that are vaporous at ambient/room temperature in a liquid state. Most processes use from ambient/room temperature to elevated temperatures. Zosel, U.S. Pat. No. 4,331,695 teaches solvent temperatures from 0° C. to 100° C. The solvent temperature is then raised to between 50° C. to 200° C. and is recirculated through the material in the same manner as a vapor degreaser. Temperatures above 0° C. not only cause the extract to pick-up more water than they would at −0° C., but it also increases the extraction of lipids, fats and waxes from the cannabis plant. What this process does not do is maintain a low temperature during the solvent recovery phase. Because of this, the amount of flavonoids and terpenes will be reduced if not lost all together. Benado, U.S. Pat. No. ______ calls for temperatures of 40° C. to 50° C. and pressures up to 250 psi. Heidlas, U.S. Pat. No. 5,405,633 teaches extraction temperatures of 10° C. to 55° C. and pressures of 10 to 30 bar. Franke, U.S. Pat. Nos. 5,281,732-5,525,746-5,739,364 and 6,248,910 call for temperatures of 60° F. to 130° F. and pressures up to 200 psi.

Corr, U.S. Pat. No. 6,676,838 uses heated hydrofluorocarbons (HFC's) vapors, not (LPG). The plant material is subjected to elevated temperatures in the extraction process. This will cause an increased amount of lipids, fats and waxes to be extracted. It will also cause the loss of the flavonoids and terpenes. There is no mention of below 0° C. processing.

Hammler, U.S. Pat. No. 8,859,793 is the first to cite hemp and cannabis as plant material to be processed. This process relies on pressure to prevent freezing of the solvent or product. Jones, U.S. Pat. Nos. 9,327,210-9,789,147 and 9,844,740 teaches a passive solvent recovery system that uses thermal gradient (heat) to evaporate the solvent in the collection reservoir and then condensed it in the cooled solvent reservoir. In addition the plant material chamber is an integral part of the extractor's structure. Safely removing and replacing the contents of the plant material chamber requires time consuming degassing of the material chamber and disassembling the entire extraction unit. McGee, U.S. Pat. Nos. 9,757,664 and 9,604,155 (marketed as Tamisum Extractors) includes automated control of liquid levels, heat and pressure. The solvent is transported by heating and cooling it. In between the solvent tank and the material chamber are two valves, with a quick disconnect in between the two valves. This is an improvement over other commercially available designs such as “Bhogart” and “Precision Extraction Solutions”, but entrapping pressurized gas or liquid solvent in between the two valves could endanger the operator when the quick coupling is opened. Also there is no meant to isolate the material chamber and collection/evaporation chamber from each other. This means to safely recharge the material chamber, both the material chamber and the collection/evaporation chambers must be out gassed to a safe level. This is very time consuming. Ayres, US Pub. No. 2017/033350 teaches the use of a co-solvent of, of CO₂ and a polar solvent. This does lower the extraction temperature, however, polar solvents are water soluble and residual water in the plant material or exposure to humidity in the atmosphere can cause water pick-up in the extract. This is harder to remove than the residual polar solvent.

### SUMMARY OF INVENTION

The prior art does not produce a finished product, only a raw product. The current invention improves on the prior art in several ways. The approach is to process at the lowest temperature and pressure as practical. Incorporated into every chamber, including the space in between any chambers isolation valves is the capability to drain, pull vacuum and purge with inert gas. Also incorporated into every chamber is a pressure relief check valve that will vent to a safe location. The process also includes the ability to homogenize the extract in the solvent recovery/distillation chamber. Also the extract is pasteurized before the residual solvent is removed. Additionally this process includes improved means not only to remove the residual solvents but also to collect QC samples.

Improvement to efficiency include maintaining the temperature of the clean solvent chamber, material chamber, used solvent chamber and the filtration chamber between 0° C. and the freezing point of the solvent being used. This saves time by the elimination of any thermal cycling to move the solvent. Also, if the solvent and plant material were both at between −30° C. and −60° C., the amount of fats, lipids and wax extracted would be greatly reduced, if not totally eliminated. This in turn greatly reduces, if not eliminates the need to dewax the oil bearing solvent.

Major improvements to efficiency include reusing the solvent by recycling it and any rinse solvent several times, before filtration or solvent recovery. Reusing the oil bearing solvent and rinse allows several smaller batches to be incorporated into one larger lot of oil bearing solvent. In addition the solvent recovery system is equipped with the means to mix/blend multiple batches of filtered oil bearing solvent into one larger homogenized lot, which improves efficiency and product quality.

Also the solvent recovery vacuum system uses back pressure regulators controls the vacuum level and preferably vacuum pumps that do not contaminate the vapors or increase the temperature of the exhaust vapors. A vacuum/compressor can increase the exhaust temperatures to as high as 350° F.

Another improvement to efficiency which is also an improvement in safety is the use of a safety valve system. This safety valve system allows for the safe removal and replacement of a material chamber without the need for time consuming out gassing.

To improve quality after the solvent distillation/oil concentration stage, the extract is transferred to a pasteurization chamber or pasteurized in transfer lines before it enters a residual solvent removal chamber. This chamber is capable of pressure or vacuum and is equipped with a high shear three or five roll mill with thermally controlled rollers.

The three or five roll mill not only improves homogenization, it causes the surface area of the extract to be repeatedly/continuously exposed to the environment which greatly speeds up the removal of the residual solvent.

Another quality improvement is to take Q.C. samples as the extract is being discharged after the point of homogenization or after the residual solvent removal. This will allow the entire lot of extract to be sampled in real time and streamline the QC process.

### BRIEF DESCRIPTION OF DRAWINGS

FIG. 1 is of a system used with solvents that are vaporous at ambient or room temperature, such as propane or butane.

FIG. 2 is of a system that would be used with solvents that are liquid at ambient or room temperature, such as ethanol or hexane.

FIG. 3a shows the upper safety valving system used on the top of removable material/reaction chamber in FIG. 1.

FIG. 3b shows the lower safety valving system that is located on the bottom of the removable material/reaction chamber in FIG. 1.

FIG. 4 Shows a removable material/reaction chamber 18 after it has been relocated to a rinse/degassing station.

FIG. 5a shows a transfer/pasteurization tank 33 that can attached to the discharge valve 10 of the vacuum evaporation system 8.

FIG. 5b shows a residual solvent removal vacuum chamber 37 attached directly to the thermally controlled vacuum evaporation system 8.

### DETAILED DESCRIPTION OF DRAWINGS

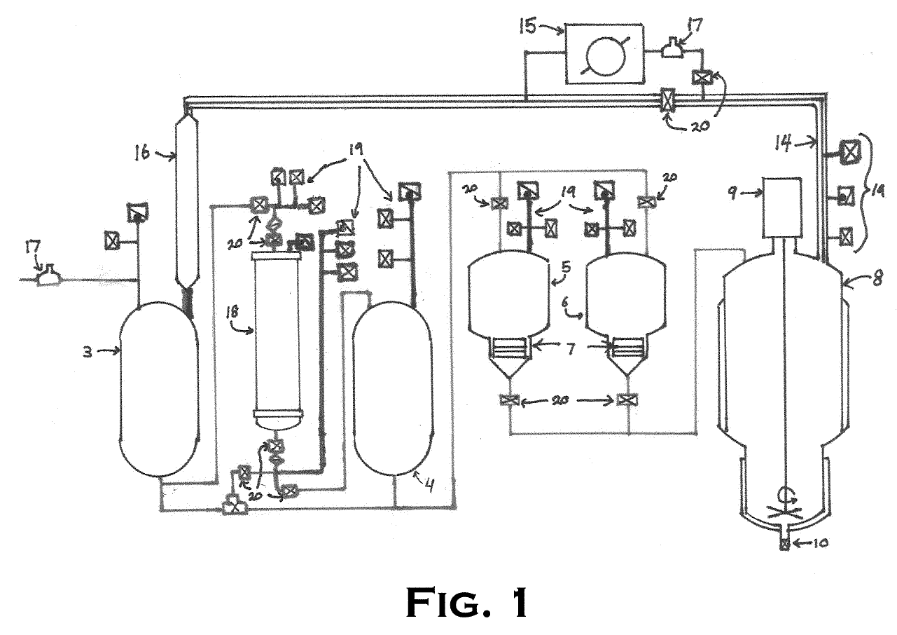

FIG. 1 is of a system used with solvents that are vaporous at ambient or room temperature, such as propane or butane. The process has four section: solvent storage and reaction, filtration, solvent recovery/distillation and homogenization, residual solvent removal/pasteurization. The solvent storage and reaction section is comprised of a clean solvent tank 3, a used solvent storage tank 4 and a removable material/reaction chamber 18. The plant material in the reaction chamber 18 is covered with solvent from the clean solvent tank 3. After a pre-determined time the oil bearing solvent is the top of the reaction chamber 18 and then transferred to the used solvent tank 4. Then the top and bottom isolation valves 20 are closed and the space in between them is evacuated and purged with inert gas to ensure it is safe to disconnect the removable reaction chamber. The removable reaction chamber 18 can then be relocated to a safe area and allowed to out gas while another reaction chamber can be installed.

After several solvent reaction and rinse cycles the oil bearing solvent in the used solvent tank is transferred to the filtration section. This section is comprised of filter tanks 5 and 6 with a multi membrane filtration module 7 and holding tanks (not shown).

After the oil bearing material has been filtered, it is transferred to the solvent recovery/distillation and homogenization section. This phase of the process is done in a thermally controlled solvent recovery system 8 that is fitted with an internal mixer 9 to mix/blend multiple batches into a homogenized lot and a discharge valve 10 at the bottom. Vacuum is applied by a non-contaminating vacuum pump 15 through a vacuum line 14. The vacuum level is controlled by a back flow vacuum regulator 17. The exhaust vapors from the vacuum pump 15 then flow through a set of condensers 16 where the vapors are chilled to a liquid state before entering the clean solvent tank 3.

Every chamber or tank can be isolated from each other with isolation valves 20. Additionally all chambers or tanks, including the space in between the isolation valves have a manifold 19 consisting of a vacuum valve, inert gas valve and an over pressure relief valve.

All of the solvent tanks 3 and 4, filtration tanks 5 and 6 and the reaction chamber 18 are refrigerated in order to maintain a constant temperature of between 0° C. to the freezing point of the solvent being used After being filtered, the oil bearing solvent is transferred to the solvent recovery/distillation and homogenization section (not shown).

FIG. 2 is of a system that would be used with solvents that are liquid at ambient or room temperature, such as ethanol or hexane. A charge of frozen plant material 1 that is in a commercially available brew or filter sack is placed into a perforated tub/canister 1b that has a hub that allows it to spin. The canister/tub 1b is located inside a material chamber 2 which includes a lid 2a that seals the chamber when closed. The plant material is reacted with solvent from the clean solvent tank 3. b When the plant material is covered with solvent, agitation/vibration can be applied to the canister/tub 1b. At a predetermined time the solvent is then transferred to the used solvent tank 4. The plant material can be rinsed with solvent from the clean solvent tank 3 at the same time the canister/tub 1b can be spun to remove most of the solvent on the plant material as the rinse solvent is being transferred to the used solvent tank 4. After the rinse cycle the plant material can be replaced with another charge of frozen plant material 1. For the next few reaction cycles, used solvent and rinse solvent from the used solvent tank 4 is used. During the rinse cycle clean solvent is used. After several charges of frozen plant material have been processed through the reaction/rinse cycle the contents of the used solvent tank are transferred to the filtration section. As in FIG. 1, this section is comprised of filter tanks 5 and 6 with a multi membrane filtration module 7 and holding tanks (not shown).

As in FIG. 1 after the oil bearing material has been filtered, it is transferred to the solvent recovery/distillation and homogenization section. This phase of the process is done in a thermally controlled solvent recovery system 8 that is fitted with an internal mixer 9 to mix/blend multiple batches into one homogenized lot and also has a discharge valve 10 at the bottom. Vacuum is applied by a non-contaminating vacuum pump 15 through a vacuum line 14. The vacuum level is controlled by a back flow vacuum regulator 17. The exhaust vapors from the vacuum pump 15 then flow through a set of condensers 16 where they are chilled to a liquid state before entering the clean solvent tank 3.

Every chamber or tank can be isolated from each other with isolation valves 20. Additionally all chambers or tanks, including the space in between the isolation valves have a manifold 19 consisting of a vacuum valve, inert gas valve and an over pressure relief valve. As in FIG. 1 all of the solvent tanks 3 and 4, filtration tanks 5 and 6 and the reaction chamber 2 and 2a are refrigerated as to maintain a constant temperature between 0° C. to the freezing point of the solvent being used.

As in FIG. 1 all of the solvent tanks 3 and 4, filtration tanks 5 and 6 and the reaction chamber 18 are refrigerated to maintain a constant temperature between 0° C. to the freezing point of the solvent being used.

After being filtered, the oil bearing solvent is transferred the solvent recovery/distillation and homogenization section (not shown).

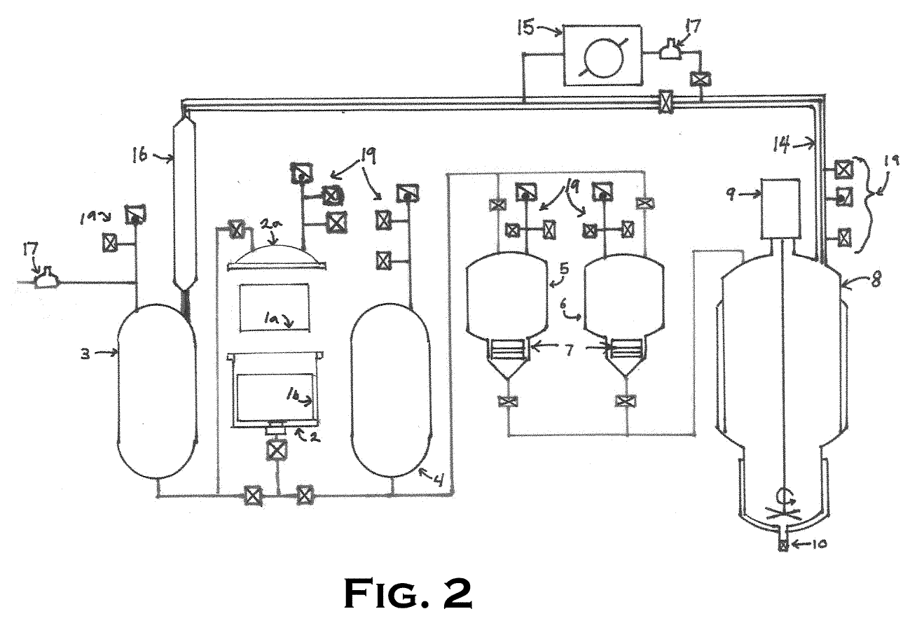

FIG. 3a shows the upper safety valving system used on the top of removable material/reaction chamber 18. The safety valving systems use an upper isolation valve 21 that allows solvent to enter the material/reaction chamber 18 and a lower isolation valve 22. In between the isolation valves is a quick disconnection device 23 that seals the upper and lower portion of the safety valving system. The top portion of the safety valving system also has an inert gas valve 24 a vacuum valve 25 and an over pressure check valve 26. The lower portion of the upper safety valving system is also the top 31 of the removable material/reaction chamber 18 and is attached using a quick disconnect 23 to make a sealed connection. There is also has an over pressure check valve 25 that can vent the removable material/reaction chamber if needed.

FIG. 3b shows the lower safety valving system that is located on the bottom of the removable material/reaction chamber 18. The lower safety valving system is a mirror of the upper safety valving system. It also has a quick disconnect 23 in between an upper and lower isolation valve 21 and 22. The lower portion has an inert gas valve 24 a vacuum valve 25 and an over pressure check valve 26. Because the clean solvent and used solvent tanks 3 and 4 are not below the isolation valves, a secondary drain valve 27 is needed to flush out the lower safety valving system. When both isolation valves 21 and 22 are closed, the secondary drain valve 27 can be opened. Then the inert gas valve 24 can be opened so pressurized inert gas can drive the residual solvent into the used solvent tank 4.

FIG. 4 Shows a removable material/reaction chamber 18 after it has been relocated to a rinse/degassing station. This view of the removable material/reaction chamber shows sight glass view ports and transportation/mounting brackets 29 not previously shown. The rinse/degassing station uses a safety valving system similar to the one previously described. The upper portion of the safety valving system has an inert gas valve 24 a vacuum valve 25 an over pressure relief valve 26 a solvent isolation valve 21 and a quick disconnect mechanism 23.

The bottom of the removable material/reaction chamber 18 is attached to a residual/rinse solvent collection chamber 30 that collects any rinse or residual solvent. On the lid/top 32 of the residual/rinse solvent chamber are two quick disconnect mechanisms 23 one connecting the top 32 to the body of the residual/rinse solvent chamber 30 and one connecting the bottom of the removable material/reaction chamber 18 to the residual/rinse chamber 30. The lid/top of the residual/rinse solvent chamber 30 has a solvent isolation valve 22 an inert gas valve 24 vacuum valve 25 and an over pressure relief check valve 26.

FIG. 5a shows a transfer/pasteurization tank 33 that can attached to the discharge valve 10 of the solvent recovery system 8 and has a sealable lid with 34 an inlet/isolation valve 35. The transfer/pasteurization tank 33 is also fitted with a discharge valve. This transfer/pasteurization tank is capable of operating up to 350° F. and above 100 psi. Once the concentrated oil has been pasteurized it can be connected to a residual solvent removal chamber (not shown) for final solvent removal.

FIG. 5b shows a residual solvent removal vacuum chamber 37 attached directly to the thermally controlled solvent recovery system 8. In this configuration the concentrated oil is transferred from the solvent recovery system 8 to the residual solvent removal vacuum chamber 37 by using a metering pump 38 to control the flow. When the concentrated oil exits the metering pump 37 it enters a thermally controlled line 39 that has a high pressure check valve (not shown) at its end. This pressurized thermally controlled line can be used for pasteurization. Inside the residual solvent removal vacuum chamber 37 is a thermally controlled high shear three or five roll mill 42. The three or five roll mill 39 mixes the concentrated oil under vacuum which increases the speed of the residual solvent removal. The finished extract is discharged into a collection vessel 40 that is sitting on an electronic scale 41. As the extract is discharged QC samples are taken at predetermined intervals by opening a vacuum interlock 43 in between the residual solvent removal vacuum chamber 37 and the vacuum sample chamber 44. A QC sample is then taken by extending and then retracting a sample tray 45 at which time the vacuum interlock 43 is closed and the vacuum sample chamber 44 is vented, opened and the sample removed.

1. A solvent based extraction process consisting of at least one clean solvent storage tank, at least one used solvent tank, at least one removable material/reaction chamber, a safety valving system, at least one filtration tank with a multi membrane filtration module connected to at least one filtered solvent holding tank, a vacuum solvent distillation/oil concentration chamber, oil bearing solvent homogenization, concentrated oil pasteurization, residual solvent removal chamber and quality control sample acquisition.

2. The process of claim 1 wherein solvent tanks, material/reaction chamber, filtration system and material being processed is maintained at temperature between 0° C. and the freezing point of the solvent being used.

3. The process of claim 1 wherein the removable material/reaction chamber and material to be processed are pre-chilled in a remote location.

4. The process of claim 1 wherein a solvent collection chamber is connected to the bottom of the removable material/reaction chamber and is fitted with two isolation valves in between a quick disconnect mechanism located in between the material/reaction chamber and the solvent collection chamber.

5. The process of claim 4 wherein the solvent collection chamber that is connected to the bottom of the removable material/reaction chamber is fitted with an over pressure relief check valve, vacuum valve and an inert gas valve that is mounted on the top of the solvent collection chamber.

6. The process of claim 1 wherein the used/oil containing solvent is reused for the material reaction phase in the removable material/reaction chamber.

7. The process of claim 1 wherein the used oil bearing solvent and oil bearing rinse solvent is reused for the reaction solvent phase in the removable material/reaction.

8. The process of claim 1 wherein the safety valving system uses two isolation valves on either side of a quick disconnect mechanism

9. The process of claim 9 wherein the space in between the isolation valves is fitted with an over pressure relief check valve and has the ability to drain, evacuate, bask fill with inert gas.

10. The process of claim 1 wherein the safety valving system is located at the top of the removable material/reaction chamber connecting the solvent storage tanks and the removable material/reaction chamber.

11. The process of claim 1 wherein the safety valving system is located at the top and bottom of the removable material/reaction chamber connecting the solvent storage tanks and the removable material/reaction chamber.

12. The process of claim 1 wherein multiple removable material/reaction chambers can be attached to the solvent tanks.

13. The process of claim 1 wherein the removable material chamber is relocated to a remote rinse/degassing station.

14. The process of claim 1 wherein the remote rinse/degassing station that is connected to a solvent recovery system.

15. The process of claim 1 wherein every tank and chamber can be isolated with the ability pull a vacuum, vent/pressurize with inert gas and is fitted with an over pressure relief check valve

16. The process of claim 1 wherein the solvent recovery/system can receive and blend several batches or lots of oil bearing solvent while running continuously.

17. The process of claim 1 wherein an internal mixer in the solvent distillation chamber, homogenizes several batches of oil bearing solvent into one homogenized lot.

18. The process of claim 1 wherein the concentrated oil is pumped into a vacuum chamber to remove the residual solvent.

10. The process of claim 1 wherein the concentrated oil is pasteurized using, but not limited to a combination of heat, ultra violet light, microwave, etc.

20. The process of claim 1 wherein the concentrate oil is pasteurized in a separate thermally controlled and pressurized transfer chamber.

21. The process of claim 1 wherein concentrated oil being pumped into a vacuum chamber to remove the residual solvents, passes through a thermally controlled line or chamber that pasteurizes the oil.

22. The process of claim 1 wherein residual solvent removal includes the use of a thermally controlled three or five roll mill.

23. The process of claim 1 wherein quality control samples are taken after the point of homogenization.

24. The process of claim 1 wherein quality control samples are taken as the extract is being discharged after the point of homogenization.

25. The process of claim 1 wherein quality control samples are taken inside the residual solvent removal vacuum chamber.

26. The process of claim 1 wherein the finished extract is pumped out of the residual solvent removal chamber using a metering pump equipped with a vacuum check valve.

27. The process of claim 1 wherein the material/reaction chamber is not removable.

28. The process of claim 1 wherein the material/reaction chamber is comprised of a chamber, a sealable lid and a perforated tub/canister mounted on a hub that can introduce centrifugal force to the reacted material to remove excess solvent.

29. The process of claim 1 wherein the vacuum solvent distillation system uses at least one non-contamination vacuum pump.

30. The process of claim 1 where in the vacuum levels are controlled by vacuum back pressure regulators.
